(19) United States
(12) Patent Application Publication
(10) Pub. No.: US 2025/0295158 A1
(43) Pub. Date: Sep. 25, 2025

(54) COMPOSITIONS COMPRISING CYS-PEPTIDES

(71) Applicant: Evonik Operations GmbH, Essen (DE)

(72) Inventors: Martin SCHILLING, Bonn (DE); Anne BENEDIKT, Frankfurt (DE); Christina JOST, Alsbach-Haehnlein (DE); Mario GOMEZ, Darmstadt (DE); Christian KESSLER, Aschaffenburg (DE)

(73) Assignee: Evonik Operations GmbH, Essen (DE)

(21) Appl. No.: 19/188,108

(22) Filed: Apr. 24, 2025

Related U.S. Application Data
(62) Division of application No. 17/996,460, filed on Oct. 18, 2022.

(30) Foreign Application Priority Data
Apr. 20, 2020 (EP) 20170403.8

(51) Int. Cl. A23L 33/18; A61K 8/44; A61K 8/64; A61Q 19/00; C12N 5/00

(52) U.S. Cl. CPC A23L 33/18; A61K 8/447; A61K 8/64; A61Q 19/00; C12N 5/0018; C12N 2500/32

(57) ABSTRACT

A composition may include at least one oligopeptide having solely alpha-peptide-bonds with one amino acid being cysteine (Cys), and free cysteine. A culture medium and may be used for culturing cells, preferably plant cells, animal cells, or mammalian cells, and a cell culture product may be manufactured using such a method.

### FIELD OF THE INVENTION

The present invention relates to compositions that are prepared by mixing at least one oligopeptide, preferably a dipeptide, with one amino acid being cysteine (Cys), and a cysteine source selected from free cysteine and optionally cystine (Cys-Cys) wherein the composition has a pH-value of at least 5, preferably of at least 6. The compositions have the advantage of improved stability/solubility compared to free cysteine/cystine and can be used to provide a source of cysteine/cystine to cells, tissues, organs and whole organisms.

Moreover, the present invention relates to biotechnological production processes. More specifically, the present invention relates to improved culture media for use in biotechnological production processes, processes employing such improved media, and to products obtained from the processes using the improved culture media.

### BACKGROUND OF THE INVENTION

Cysteine is an important amino acid that is used in various applications, from human nutrition, cosmetics to cell and tissue culture processes. It is a building block for proteins and for glutathione, which is an important cellular antioxidant and involved in the regulation of redox homeostasis. Various health benefits have been demonstrated for oral nutritional cysteine/cystine supplementation (reviewed by Plaza et al., Molecules 2018, 23, 575)

Cysteine/cystine is also present in cell culture medium formulations. Although cysteine is not an essential amino acid, limitations in cell culture processes for biologics production can lead to severe loss of viability and productivity. On the other hand, overdosing of cysteine can lead to toxic effects (Ritacco et al., Biotechnol Prog., 2018, Vol. 34, No. 6).

A major problem in the formulation of cysteine in highly concentrated liquid nutrient solutions for various application is that it rapidly oxidizes to cystine in the presence of oxygen and cystine has a low solubility of <1 mM in a pH range between 3 and 9 (Carta et al., J. Chem. E ng. Data 1996, 41, 414-417).

A solution to this problem is the use of well soluble Cys-dipeptides such as (Ala-Cys)₂ and (Lys-Cys)₂ as a source of cysteine/cystine. Those peptides have found use in cell culture, parenteral nutrition and have been investigated for cosmetic applications (see references below).

However, due to additional synthesis and purification steps required to produce dipeptides, they are significantly more expensive than free cysteine and cystine. Therefor there is still a need for more cost-efficient, highly concentrated cysteine/cystine formulations. Depending on the nature of the other amino acid in the peptide, the solubility increase is sometimes not high enough, which poses further restrictions on formulation possibilities.

EP2561065B1 discloses concentrated feed comprising concentrations of cysteine and tyrosine that support maximal cell growth and/or protein, or viral production while avoiding the problems caused by their limited solubility and stability.

US2013/0130317 A1 describes a method for culturing animal cells having an ability to produce a substance, which comprises culturing the animal cells in a medium supplemented with an oligopeptide having one or more L-cysteines and excluding glutathione.

For the same reason, Cys-peptides have also been evaluated as alternatives to cysteine in parenteral nutrition (Pollack et al., Zeitschrift für Ernährungswissenschaften 1989, 28: 191-200) and cosmetic applications (Tseng et al., J. Agric. Food Chem. 2015, 63: 6181-6188).

However, synthetic peptides are significantly more expensive than amino acids and lower cost solutions to stabilize cysteine against precipitation are therefore desirable. Reducing agents can be used to prevent cystine formation from cysteine to a certain extent when oxygen can be physically excluded (reference) but are often not desired. Also, this approach does not work in the presence of high oxygen concentrations and supply, such as an aerated bioreactor environment used for cell culture.

For some Cys-peptides (e.g. (Ala-Cys)₂), solubility is still not high enough to prepare highly concentrated stock or feed solutions. However, in modern cell culture bioprocessing, highly concentrated solutions of amino acids and peptides are important to intensify the processes and to avoid excessive liquid processing (e.g. in perfusion systems), which can reduce flexibility and productivity, complicates downstream processing and comes with a higher environmental burden.

Similar problems occur in aqueous food and cosmetic products or in nutrient solutions for parenteral nutrition.

### SUMMARY OF THE INVENTION

The above shortcomings of highly concentrated cysteine containing liquid formulations are addressed by the present invention. The invention is defined by the terms of the appended independent claims. Preferred embodiments of the invention are defined by the dependent claims.

Surprisingly it was found, that highly concentrated solutions of cysteine can be stabilized against oxidative precipitation by addition of cysteine containing oligopeptides, dipeptides, and/or their disulfides. Even more surprisingly, respective mixtures even resulted in increased solubility of the respective Cys-oligopeptide in some cases. It was also found that cystine can be solubilized by addition of Cys-peptides in the presence of small amounts of cysteine (or other thiols, not investigated).

Obtained compositions represent stable cysteine forms that can be prepared at significantly lower cost per cysteine-equivalent compared to dipeptides alone and thus enable additional more cost sensitive applications than the current ones. An additional advantage is, that they can be used in applications where a certain amount of free cysteine is required, e.g. when the hydrolysis rate of the oligo- or dipeptide to release cysteine or cystine becomes rate limiting.

The compositions can be prepared by mixing defined molar ratios of Cys-oligopeptide or Cys-dipeptide with cysteine or by mixing defined molar ratios of Cys-dipeptide with cysteine and cystine. The preferred molar ratio of the oligopeptide bound cysteine to the free cysteine is 10 or lower, preferably 4 or lower, more preferably 2 or lower, more preferable 1 or lower, more preferable 0.5 or lower, most preferable 0.2 or lower. The mixtures can be in the form of solids (crystalline powders, agglomerates etc) or aqueous solutions. In the case of aqueous solutions, cysteine is added in a concentration of at least 1 mM, preferable at least 10 mM, more preferable at least 50 mM and most preferable at least 100 mM and the dipeptide is added at the appropriate molar ratio described above.

The compositions according to the present invention can also be a component part of a cosmetic product, a nutritional supplement, a nutrient solution for clinical nutrition, or a cell or tissue culture medium (basal, feed or perfusion medium).

The invention thus relates to compositions prepared by mixing at least one oligopeptide, preferably at least one dipeptide, with one amino acid being cysteine (Cys), and a cysteine source selected from free cysteine and optionally cystine (Cys-Cys). The invention thus relates to a culture medium comprising the composition.

The invention further relates to the use of a culture medium of the invention for culturing cells, preferably plant cells, animal cells or mammalian cells.

Another aspect of the invention relates to a method of manufacturing a cell culture product comprising the steps of (i) providing a cell capable of producing said cell culture product; (ii) contacting said cell with a culture medium according to the invention; and (iii) obtaining said cell culture product from said culture medium or from said cell.

Preferred embodiments of the invention are described in further detail in the following detailed description of the invention.

### BRIEF DESCRIPTION OF THE FIGURES

FIG. 1 depicts the formation of precipitates in a 100 mM cysteine solution at pH=7 at 37° C. over 24 hours in the presence of increasing concentrations of (Lys-Cys)₂.

FIG. 2 shows reduction of turbidity of a 50 mM (Ala-Cys)₂ suspension by adding increasing amounts of cysteine.

FIG. 3 shows stabilization of a 100 mM cysteine solution against oxidative precipitation by addition of various concentrations of (Lys-Cys)₂.

FIG. 4: shows the stabilizing effect against oxidative precipitation provided by addition of various concentrations of Cys-peptides to a 50 mM cysteine solution.

FIG. 5 illustrates the stabilizing effect against oxidative precipitation provided by addition of various concentrations of Cys-peptides to a 25 mM cysteine solution.

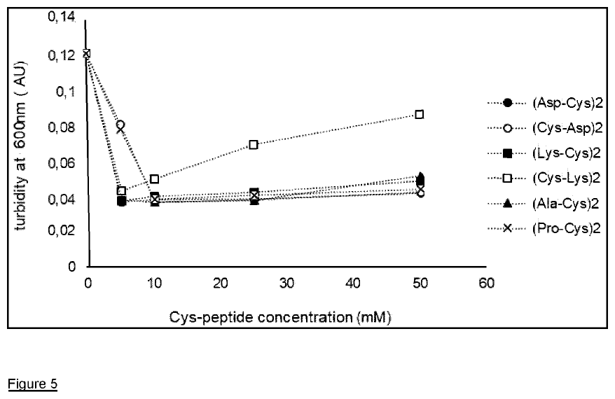

### DETAILED DESCRIPTION OF THE INVENTION

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention, for instance “free” cysteine, is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The present invention generally relates to a composition comprising at least one oligopeptide with one amino acid being cysteine (Cys) and (as a cysteine source) free cysteine. Optionally free cysteine and free cystine (Cys-Cys) may be comprised. The terms free cysteine and free cystine (Cys-Cys) shall include the salts or hydrate forms of cysteine and cystine.

Preferably the oligopeptide is a dipeptide.

A “peptide” shall be understood as being a molecule comprising at least two amino acids covalently coupled to each other by alpha-peptide bonds (R¹—CO—NH—R²).

A “polypeptide” shall be understood as being a molecule comprising more than twenty amino acids covalently coupled to each other by peptide bonds (R¹—CO—NH—R²).

An “oligopeptide” shall be understood as being a molecule comprising less than twenty, preferably two to ten amino acids covalently coupled to each other solely by alpha-peptide-bonds (R¹—CO—NH—R²). Glutathione, a Glu-Cys-Gly tripeptide, which contains a gamma-peptide-bond and an alpha-peptide-bond is therefore excluded.

A “dipeptide” shall be understood as being a molecule comprising two amino acids covalently coupled to each other by an alpha-peptide-bond (R¹—CO—NH—R²).

The expression “Xxx”, when used herein in connection with an amino acid, shall be understood as referring to any natural amino acid as defined in the following.

An “amino acid”, in the context of the present invention, shall be understood as being a molecule comprising an amino functional group (—NH₂) and a carboxylic acid functional group (—COOH), along with a side-chain specific to the respective amino acid. In the context of the present invention, both alpha- and beta-amino acids are included. Preferred amino acids of the invention are alpha-amino acids, in particular the 20 “natural amino” acids including cystine as follows:

| | |
|---|---|
| Alanine | (Ala/A) |
| Arginine | (Arg/R) |
| Asparagine | (Asn/N) |
| Aspartic acid | (Asp/D) |
| Cysteine | (Cys/C) |
| Cystine | (Cyss/C2) |
| Glutamic acid | (Glu/E) |
| Glutamine | (Gln/Q) |
| Glycine | (Gly/G) |
| Histidine | (His/H) |
| Isoleucine | (Ile/I) |
| Leucine | (Leu/L) |
| Lysine | (Lys/K) |
| Methionine | (Met/M) |
| Phenylalanine | (Phe/F) |
| Proline | (Pro/P) |
| Serine | (Ser/S) |
| Threonine | (Thr/T) |
| Tryptophan | (Trp/W) |
| Tyrosine | (Tyr/Y) |
| Valine | (Val/V) |

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention (for instance “free cysteine”), is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The expression “N-acylated”, with reference to a chemical compound, such as an amino acid, shall be understood as meaning that the N-acylated compound is modified by the addition of an acyl group to a nitrogen functional group of said compound. Preferably, the acyl group is added to the alpha-amino group of the amino acid.

In the context of this invention, Cys-peptides forming a disulfide bond via oxidized cysteine residues, shall be described by (Xxx-Cys)₂. The peptides may also be present as salts or in hydrate form. Such disulfide bond mediated dimers of Cys-dipeptides, for instance (Xxx-Cys)₂, are still considered as a dipeptides in the sense of the invention.

Preferably, the composition has a pH-value at 25° C. of at least 5 or preferred of at least 6.

In an advantageous configuration of the present invention, a molar ratio of the peptide-bound cysteine to free cysteine is between 0.1 and 10, preferably between 0.2 and 4 or lower, most preferable between 0.5 and 2 In a preferred embodiment, the oligo- or dipeptides are either in a reduced state (=free thiol) or oxidized state (=disulfide bonded), preferably in an oxidized state

In an alternative embodiment, the composition comprises a mixed disulfide of the dipeptide and the cysteine source.

In a preferred embodiment of the present invention, the oligo- or dipeptide further comprises one or more natural amino acids with a solubility of at least >10 g/l at a pH range between pH 6 and pH 9 and is preferably selected from glycine (Gly), alanine (Ala), serine (Ser), proline (Pro), aspartic acid (Asp), glutamic acid (Glu), lysine (Lys) orarginine (Arg).

It is preferred, when said oligopeptide is a dipeptide which is Xxx-Cys or Cys-Xxx, wherein Xxx is a natural amino acid, preferably the dipeptide preferably being Asp-Cys, Cys-Asp, Lys-Cys, Cys-Lys, Ala-Cys or Pro-Cys. In a preferred configuration, said dipeptide is present in said culture medium at a concentration of at least 1 mM, preferably at least 10 mM, more preferably at least 50 mM, more preferably at least 100 mM. At such high concentrations, the composition according to the present invention provides the advantage that the cysteine-containing dipeptides stabilize cysteine against oxidative precipitation.

In preferred embodiments, the oligo- or dipeptide is not N-acylated. N-acylation is known to improve heat stability of certain dipeptide; however, it has been found that N-acylated dipeptides may also lead to inferior viable cell density and viability.

The present invention is also directed to a cosmetic product, a nutritional supplement or nutrient solution for clinical nutrition comprising the composition according to the present invention.

The cosmetic product may be a shampoo, conditioner, lotion, cream or other formulations used to treat skin or hair. Nutritional supplements may be in liquid form, such as syrups or shots, or in solid form, such as capsules, soft-gels, gummies. The compositions can also be part of nutrient solutions for clinical enteral or parenteral nutrition, e.g. part of an amino acid solution such as Aminoven (Fresenius Kabi).

Moreover, the present invention also refers to a cell or tissue culture medium.

Another subject of the present invention is directed to a cell or tissue culture medium comprising the composition according to the present invention, which further comprises at least one carbohydrate, at least one free amino acid, at least one inorganic salt, a buffering agent and/or at least one vitamin. In a particularly preferred embodiment, the culture medium comprises all of at least one carbohydrate, at least one free amino acid, at least one inorganic salt, a buffering agent and at least one vitamin.

In one embodiment of the invention, the culture medium does not contain a growth factor. In accordance with this embodiment, the oligo- or dipeptide of the invention may be used instead of a growth factor for promoting growth and/or proliferation of the cells in culture. In another embodiment of the invention, the culture medium does not contain any lipids.

According to another embodiment of the invention, the culture medium is in liquid form, in form of a gel, a powder, a granulate, a pellet or in form of a tablet.

In preferred embodiments, the culture medium of the invention is a defined medium, or a serum-free medium. For example, the compositions of the invention may be supplemented to the CHOMACS CD medium of Miltenyi Biotech (Bergisch Gladbach, Germany), to the PowerCHO-2 CD medium available from LONZA (Basel, Switzerland), the Acti-CHO P medium of PAA (PAA Laboratories, Pasching, Austria), the Ex-Cell CD CHO medium available from SAFC, the SFM4CHO medium and the CDM4CHO medium of ThermoFisher (Waltham, USA). The dipeptides of the invention may also be supplemented to DMEM medium (Life Technologies Corp., Carlsbad, USA). The invention, however, is not limited to supplementation of the above media.

In other preferred embodiments, the culture medium is a liquid medium in 2-fold, 3-fold, 3.33-fold, 4-fold, 5-fold or 10-fold concentrated form (volume/volume), relative to the concentration of said medium in use. This allows preparation of a “ready-to-use” culture medium by simple dilution of the concentrated medium with the respective volume of sterile water. Such concentrated forms of the medium of the invention may also be used by addition of the same to a culture, e.g., in a fed-batch cultivation or perfusion process.

The cell culture medium (cell or tissue culture basal, feed or perfusion medium) of the present invention may preferably contain all nutrients required for sustained growth and product formation.

Recipes for preparing culture media, in particular cell culture media, are well known to the person skilled in the art (see, e.g., Cell Culture Technology for Pharmaceutical and Cell-Based Therapies, Ozturk and Wei-Shou Hu eds., Taylor and Francis Group 2006). Various culture media are commercially available from various sources.

The culture media of the invention may preferably include a carbohydrate source. The main carbohydrate used in cell culture media is glucose, routinely supplemented at 5 to 25 mM. In addition, any hexose, such as galactose, fructose, or mannose or a combination may be used.

The culture medium typically may also include at least the essential amino acids (i.e., His, lie, Leu, Lys, Met, Phe, Thr, Try, Val) as well as non-essential amino acids. A non-essential amino acid is typically included in the cell culture medium if the cell line is not capable of synthesizing the amino acid or if the cell line cannot produce sufficient quantities of the amino acid to support maximal growth. In addition, mammalian cells can also use glutamine as a major energy source. Glutamine is often included at higher concentrations than other amino acids (2-8 mM). However, as noted above, glutamine can spontaneously break down to form ammonia and certain cell lines produce ammonia faster, which is toxic.

The culture media of the invention may preferably comprise salts. Salts are added to the cell culture medium to maintain isotonic conditions and prevent osmotic imbalances. The osmolality of a culture medium of the invention is about 300 mOsm/kg, although many cell lines can tolerate an approximately 10 percent variation of this value or higher. The osmolality of some insect cell cultures tend to be higher than 300 mOsm/kg, and this may be 0.5 percent, 1 percent, 2 to 5 percent, 5-10 percent, 10-15 percent, 15-20 percent, 20-25 percent, 25-30 percent higher than 300 mOsm/kg. The most commonly used salts in cell culture medium include Na⁺, K⁺, Mg²⁺, Ca²⁺, Cl⁻, SO₄²⁻, PO₄³⁻, and HCO₃˜(e.g., CaCl₂), KCl, NaCl, NaHCO₃, Na₂HPO₄).

Other inorganic elements may be present in the culture medium. They include Mn, Cu, Zn, Mo, Va, Se, Fe, Ca, Mg, Si, and Ni. Many of these elements are involved in enzymatic activity. They may be provided in the form of salts such as CaCl₂), Fe(NO₃)₃, MgCl₂, MgSO₄, MnCl₂, NaCl, NaHCO₃, Na₂HPO₄, and ions of the trace elements, such as, selenium, vanadium and zinc. These inorganic salts and trace elements may be obtained commercially, for example from Sigma (Saint Louis, Missouri).

The culture media of the invention preferably comprise vitamins. Vitamins are typically used by cells as cofactors. The vitamin requirements of each cell line vary greatly, although generally extra vitamins are needed if the cell culture medium contains little or no serum or if the cells are grown at high density. Exemplary vitamins preferably present in culture media of the invention include biotin, choline chloride, folic acid, i-inositol, nicotinamide, D-Ca⁺⁺-pantothenate, pyridoxal, riboflavin, thiamine, pyridoxine, niacinamide, A, B₆, B₁₂, C, D₃, E, K, and p-aminobenzoic acid (PABA).

Culture media of the invention may also comprise serum. Serum is the supernatant of clotted blood. Serum components include attachment factors, micronutrients (e.g., trace elements), growth factors (e.g., hormones, proteases), and protective elements (e.g., antitoxins, antioxidants, antiproteases). Serum is available from a variety of animal sources including human, bovine or equine serum. When included in cell culture medium according to the invention, serum is typically added at a concentration of 5-10% (vol.). Preferred cell culture media are serum-free.

To promote cell growth in the absence or serum or in serum reduced media, one or more of the following polypeptides can be added to a cell culture medium of the invention: for example, fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, any cytokine, such as interleukins 1, 2, 6, granulocyte stimulating factor, leukocyte inhibitory factor (LIF), etc.

In other embodiments, the cell culture medium does not comprise polypeptides (i.e., peptides with more than 20 amino acids).

One or more lipids can also be added to a cell culture medium of the invention, such as linoleic acid, linolenic acid, arachidonic acid, palmitoleic acid, oleic acid, polyenoic acid, and/or fatty acids of 12, 14, 16, 18, 20, or 24 carbon atoms, each carbon atom branched or unbranched), phospholipids, lecithin (phophatidylcholine), and cholesterol. One or more of these lipids can be included as supplements in serum-free media. Phosphatidic acid and lysophosphatidic acid stimulate the growth of certain anchorage-dependent cells, such as MDCK, mouse epithelial, and other kidney cell lines, while phosphatidylcholine, phosphatidylethanolamine, and phosphatidylinositol stimulate the growth of human fibroblasts in serum-free media. Ethanolamine and cholesterol have also been shown to promote the growth of certain cell lines. In certain embodiment, the cell culture medium does not contain a lipid.

One or more carrier proteins, such as bovine serum albumin (BSA) or transferrin, can also be added to the cell culture medium. Carrier proteins can help in the transport of certain nutrients or trace elements. BSA is typically used as a carrier of lipids, such as linoleic and oleic acids, which are insoluble in aqueous solution. In addition, BSA can also serve as a carrier for certain metals, such as Fe, Cu, and Ni. In protein-free formulations, non-animal derived substitutes for BSA, such as cyclodextrin, can be used as lipid carriers.

One or more attachment proteins, such as fibronectin, laminin, and pronectin, can also be added to a cell culture medium to help promote the attachment of anchorage-dependent cells to a substrate.

The cell culture medium can optionally include one or more buffering agents. Suitable buffering agents include, but are not limited to, N-[2-hydroxyethyl]-piperazine-N′-[2-ethanesulfonic acid](HEPES), MOPS, MES, phosphate, bicarbonate and other buffering agents suitable for use in cell culture applications. A suitable buffering agent is one that provides buffering capacity without substantial cytotoxicity to the cells cultured. The selection of suitable buffering agents is within the ambit of ordinary skill in the art of cell culture.

Polyanionic or polycationic compounds may be added to the culture medium to prevent the cells from clumping and to promote growth of the cells in suspension.

In a preferred embodiment, the culture medium is in liquid form. The culture medium, however, can also be a solid medium, such as a gel-like medium, e.g. an agar-agar-, carrageen- or gelatine-containing medium (powders, aggregated powders, instantized powders etc.). Preferably, the culture medium is in sterile form.

The culture medium of the present invention can be in concentrated form. It may be, e.g., in 2- to 100-fold concentrated form, preferably in 2-fold, 3-fold, 3.33-fold, 4-fold, 5-fold, 10-fold, 20-fold, 50-fold or 100-fold (relative to a concentration that supports growth and product formation of the cells).

Such concentrated culture media are helpful for preparing the culture medium for use by dilution of the concentrated culture medium with an aqueous solvent, such as water. Such concentrated culture media may be used in batch culture but are also advantageously used in fed-batch or continuous cultures, in which a concentrated nutrient composition is added to an ongoing cultivation of cells, e.g., to replenish nutrients consumed by the cells during culture.

In other embodiments of the invention, the culture medium is in dry form, e.g., in form of a dry powder, or in form of granules, or in form of pellets, or in form of tablets.

The present invention also relates to the use of a culture medium of the invention for culturing cells. Another aspect of the invention relates to the use of a culture medium of the invention for producing a cell culture product.

A preferred embodiment of the invention relates to the use of a culture medium according to the invention for culturing animal cells or plant cells, most preferred mammalian cells. In specific embodiments the cells to be cultured are CHO cells, COS cells, VERO cells, BHK cells, HEK cells, HELA cells, AE-1 cells, insect cells, fibroblast cells, muscle cells, nerve cells, stem cells, skin cells, endothelial cells and hybridoma cells. Preferred cells of the invention are CHO cells and hybridoma cells. Most preferred cells of the invention are CHO cells. Particularly preferred CHO cells of the invention are CHO DG44 and CHO DP12 cells.

Also included in the scope of the present invention is a method of culturing cells, said method comprising contacting said cells with a cell culture medium according to the invention. In one embodiment of the invention, the method of culturing cells comprises contacting the cell with a basal culture medium under conditions supporting the cultivation of the cell and supplementing the basal cell culture medium with a concentrated medium according to the present invention. In preferred embodiments, the basal culture medium is supplemented with the concentrated feed or medium on more than one day.

Another aspect of the invention relates to a method of producing a culture medium according to the invention, wherein said culture medium comprises a composition according to the invention.

Methods of producing a culture medium according to the invention comprise at least one step of adding the composition of the invention to the culture medium. Likewise, an aspect of the invention relates to the use of a composition of the invention for producing a cell culture medium.

Another aspect of the invention relates to a method of modifying a culture medium, wherein said modifying of said culture medium comprises addition of the composition of the invention to said culture medium.

Another aspect of the invention relates to a method of producing a liquid culture medium, said method comprising providing solid medium according to the invention, e.g., in form of a dry powder, or in form of granules, or in form of pellets, or in form of tablets; and dissolving said solid culture medium in an aqueous medium, such as water.

Another aspect of the invention relates to the use of a composition according to the invention in a culture medium for culturing cells. Another aspect of the invention relates to the use of a composition according to the invention for cell culture.

The invention also relates to methods of manufacturing a cell culture product comprising the steps of (i) providing a cell capable of producing said cell culture product; (ii) contacting said cell with a culture medium of the invention; and (iii) obtaining said cell culture product from said culture medium or from said cell. Likewise, the present invention relates to the use of a composition according to the invention for manufacturing a cell culture product.

In preferred methods, the cell culture product is a therapeutic protein, a diagnostic protein, a polysaccharide, such as heparin, an antibody, a monoclonal antibody, a growth factor, an interleukin, virus, virus-like particle or an enzyme Cultivation of cells, according to the invention can be performed in batch culture, in fed-batch culture or in continuous culture.

### Items

The invention is concerned with the following items:
1. A composition, comprising at least one oligopeptide comprising solely alpha-peptide-bonds with one amino acid being cysteine (Cys), and free cysteine.
2. The composition according to item 1, comprising free cysteine and free cystine (Cys-Cys).
3. The composition according to item 1 or 2, wherein the oligopeptide is a dipeptide.
4. The composition according to any of the preceding items, wherein the composition has a pH-value at 25° C. of at least 5, preferably of at least 6.
5. The composition according to any of the preceding items, wherein a molar ratio of the oligopeptide bound cysteine to the free cysteine is from about 0.1 to 10, preferably from about 0.2 and 4; most preferable from about 0.5 to 2.
6. The composition according to any of the preceding items, wherein the oligopeptide is either in a reduced state (=free thiol) or in an oxidized state (=disulfide bonded), preferably in an oxidized state.
7. The composition according to any of the preceding items, wherein the oligopeptide is a dipeptide and is Xxx-Cys or Cys-Xxx, wherein Xxx is a natural amino acid.
8. The composition according to any of the preceding items, wherein the oligopeptide further comprises one or more natural amino acids with a solubility of at least 10 g/l at 25° C. at a pH range from pH 6 to pH 9; preferably selected from glycine (Gly), alanine (Ala), serine (Ser), proline (Pro), aspartic acid (Asp), glutamic acid (Glu), lysine (Lys) or arginine (Arg), more preferably selected from Ala, Pro, Asp, Lys.
9. The composition according to any of the preceding items, wherein the oligopeptide is a dipeptide and is Asp-Cys, Cys-Asp, Lys-Cys, Cys-Lys, Ala-Cys or Pro-Cys, most preferable Lys-Cys or Cys-Lys.
10. The composition according to any of t items 1 to 6 or 8, wherein the oligopeptide is a dipeptide and is (Xxx-Cys)₂ or (Cys-Xxx)₂, wherein Xxx is a natural amino acid.
11. The composition according to item 10, wherein the oligopeptide is a dipeptide and is selected from (Asp-Cys)₂, (Cys-Asp)₂, (Lys-Cys)₂, (Cys-Lys)₂, (Ala-Cys)₂, (Pro-Cys)₂, (Lys-Cys)₂ and (Cys-Lys)₂.
12. The composition according to items 10 or 11, wherein (Lys-Cys)₂ and/or (Cys-Lys)₂ are comprised at a concentration of 1 to 100 mM and cysteine is comprised at a concentration of 1 to 100 mM.
13. The composition according to items 10 or 11, wherein (Ala-Cys)₂, (Asp-Cys)₂, (Cys-Asp)₂ (Pro-Cys)₂ are comprised at a concentration of 20 to 60 mM and cysteine is comprised at a concentration of 1 to 100 mM.
14. The composition according to any of the preceding items, wherein cysteine is added to an aqueous solution at a concentration of at least 1 mM, preferably at least 10 mM, more preferable at least 25 mM, most preferably at least 50 mM.
15. A cosmetic product, nutritional supplement, nutrient solution for clinical nutrition, comprising the composition according to any of items 1 to 14.
16. A cell culture medium comprising a composition according to any of items 1 to 14, said culture medium further comprising at least one carbohydrate, and/or at least one additional free amino acid, and/or at least one inorganic salt, and/or a buffering agent and/or at least one vitamin.
17. The culture medium according to item 16, wherein said culture medium is in liquid form, in form of a gel, a powder, a granulate, a pellet or in the form of a tablet.
18. The culture medium according items 16 or 17, wherein said culture medium is in 2- to 100-fold concentrated form, preferably in 2-fold, 3-fold, 3.33-fold, 4-fold, 5-fold, 10-fold or 100-fold concentrated form, relative to the concentration of the culture medium in use.
19. Use of a culture medium according to any of items 16 to 18 for culturing cells, preferably as an aqueous stock or feed solution.
20. Use of item 19, wherein said cells are animal cells or plant cells, preferably mammalian cells.
21. Use of item 19 or 20, wherein said cells are selected from the list consisting of CHO cells, COS cells, VERO cells, BHK cells, HEK cells, HE LA cells, AE-1 cells, insect cells, fibroblast cells, muscle cells, nerve cells, stem cells, skin cells, endothelial cells, immune cells such as NK or T-cells and hybridoma cells.
22. Method of manufacturing a cell culture product comprising the steps of
   providing a cell capable of producing said cell culture product;
   contacting said cell with a culture medium of any one of items 16 to 18; and
   obtaining said cell culture product from said culture medium or from said cell.
23. Method of item 22, wherein said cell culture product is selected from the group consisting of therapeutic protein, diagnostic protein, polysaccharide, such as heparin, antibody, monoclonal antibody, growth factor, interleukin, virus, virus-like particle and enzyme.

### EXAMPLES

### Example 1: Stabilization of Cysteine Against Precipitation by Addition of Cys-Dipeptide (Lys-Cys)₂

Solutions of substances and dilutions thereof were prepared in 100 mM phosphate buffer, pH=7 in all experiments in all examples. Precipitation could be avoided in a 100 mM solution of cysteine in a 100 mM phosphate buffer at pH=7 by the addition of suitable concentrations of (Lys-Cys)₂.

Turbidity caused by precipitation was measured photometrically at 600 nm in a microtiter plate-based assay. Liquid volume per well was 200 μl and measurements were conducted over 24 hours at 37° C. in a microplate reader (Tecan). The plates were not covered with a lid.

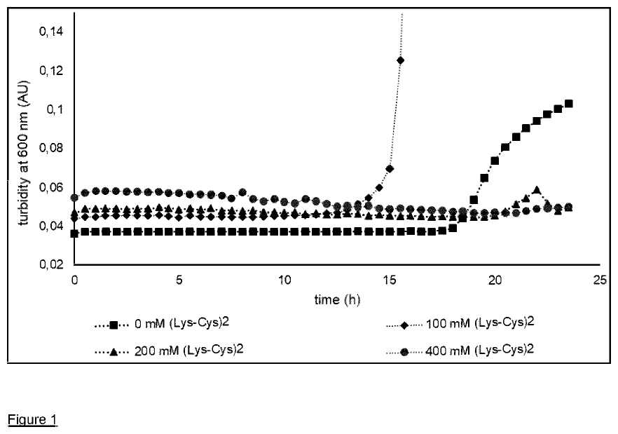

When 100 mM solutions of cysteine were prepared with 0, 100, 200 or 400 mM (Lys-Cys)_{2×2} HCl at pH=7, a surprising result was obtained. Precipitation could be seen and measured in the absence of added peptide. However, while 100 mM of (Lys-Cys)₂ even accelerated precipitation, 200 mM and 400 mM (Lys-Cys)₂ effectively prevented precipitation. The precipitates could be observed visually and measured via turbidimetry (see FIG. 1).

FIG. 1 shows formation of precipitates in a 100 mM cysteine solution at pH=7 at 37° C. over 24 hours in the presence of increasing concentrations of (Lys-Cys)₂.

### Example 2: Solubilization of Cystine by Addition of (Lys-Cys)₂ and Cysteine

20 ml of the compositions described in Table 1 were prepared in 100 mM phosphate buffer at pH=7 and incubated in closed 50 ml polypropylene tubes (Falcon) for 24 h. When adding (Lys-Cys)₂ in sufficient concentrations, clear solutions were obtained, whereas the mixtures remained turbid suspensions at lower concentrations.

TABLE 1
Millimolar concentrations of mixture components prepared in 100
mM phosphate buffer and incubated for 24 hours at 25° C.
with shaking. Visual inspection was carried out the next day.

| | Mixture 1 | Mixture 2 | Mixture 3 | Mixture 4 | Mixture 5 | Mixture 6 |
|---|---|---|---|---|---|---|
| Cystine | 1.30 | 1.30 | 1.30 | 1.30 | 1.30 | 1.30 |
| (Lys-Cys)₂ | 0.00 | 0.00 | 0.41 | 0.82 | 1.63 | 3.27 |
| Cysteine | 0.00 | 0.13 | 0.13 | 0.13 | 0.13 | 0.13 |
| Visual | suspension | suspension | suspension | suspension | solution | solution |
| inspection | | | | | | |

### Example 3: Solubilization of (Ala-CVs)₂ by Addition of Cysteine

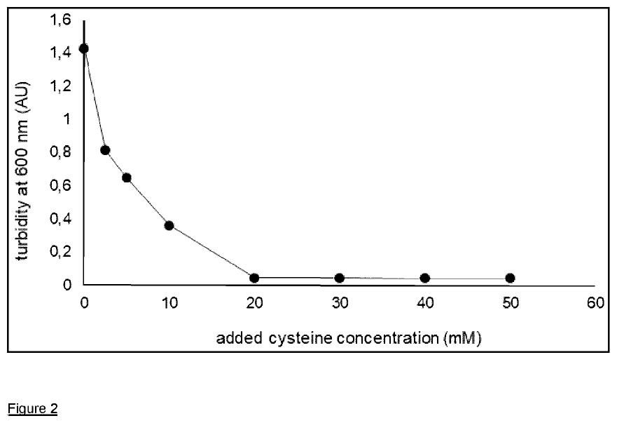

The maximum solubility of (Ala-Cys)₂ is below 50 mM at pH=7 and 37° C. Turbid suspensions of 50 mM (Ala-Cys)₂ in phosphate buffer at pH=7 were prepared and increasing amounts of cysteine were added. The mixtures were incubated at 37° C. in a microtiter plate (liquid volume per well was 200 μl) and turbidity at 600 nm was measured after 30 minutes. At concentrations of 20 mM of cysteine and higher, (Ala-Cys)₂ was fully solubilized and no further reduction of turbidity was measured. Solutions were visually transparent (see FIG. 2).

FIG. 1 shows reduction of turbidity of a 50 mM (Ala-Cys)₂ suspension by adding increasing amounts of cysteine.

### Example 4: Stabilization of a 100 mM Cysteine Solution Against Precipitation by Addition of Cys-Dipeptide (Lys-Cys)₂ Under Oxidizing Conditions

An experiment as described in example 1 was conducted in a microtiter plate. After two hours of incubation at 37° C., 14 μl of a 5 weight % H₂O₂ solution were added to each well to accelerate oxidation and evaluate stabilization under fully oxidized conditions. Samples containing only cysteine became turbid directly after addition of H₂O₂, which was most likely caused by the formation of cystine. For the samples containing (Lys-Cys)₂ no immediate increase in turbidity was observed. Turbidity measurements in the microplate reader were continued subsequently. Results are shown in Figure. Turbidity increase was strongly reduced with increasing concentrations of (Lys-Cys)₂ and no precipitate was observed when 400 mM (Lys-Cys)₂ were added.

FIG. 2 shows stabilization of a 100 mM cysteine solution against oxidative precipitation by addition of various concentrations of (Lys-Cys)₂. The mixtures were incubated in a microplate for 2 hours at 37° C. and turbidity was measured photometrically. H₂O₂ was subsequently added to accelerate oxidation and evaluate stability against precipitation under fully oxidized conditions. Turbidity measurement was continued directly after H₂O₂ addition. With cysteine alone, the solution turned directly and visibly turbid, when H₂O₂ was added. With increasing amounts of added (Lys-Cys)₂, the amount of precipitate measured via turbidity was reduced. At 400 mM (Lys-Cys)₂, the mixture remained completely clear during the cause of the measurement.

### Example 5: Stabilization of 25 mM and 50 mM Cysteine Solutions Against Precipitation by Addition of Various Cys-Dipeptides Under Oxidizing Conditions

To evaluate if the above described stabilization is also possible by addition of other Cys-dipeptides, experiments as described in example 4 were conducted with the exception that reduced cysteine concentrations of 50 mM and 25 mM were used and that the concentrations of added Cys-dipeptides were 0 (=control), 5, 10, 25 and 50 mM. The effect of (Asp-Cys)₂, (Cys-Asp)₂, (Lys-Cys)₂, (Cys-Lys)₂(Ala-Cys)₂ and (Pro-Cys)₂ was evaluated. All Cys-peptides were able to prevent precipitation from the cysteine solutions after addition of H₂O₂. Differences in stabilization efficiency were observed between the various Cys-Peptides at cysteine concentrations of 50 mM. The Cys-dipeptides containing lysine residues were the most efficient stabilizers and a molar Cys-peptide to cysteine ratio between 1:2 and 1:5 provided sufficient stability. The results of turbidity measurements with 50 mM cysteine after 4.5 hours are shown in Table 2

TABLE 2
Turbidity of a 50 mM cysteine solution mixed with various concentrations of Cys-
dipeptides. Turbidity was measured at 600 nm (AU) after 4.5 hours

| Cys-Peptide | | | | | | |
|---|---|---|---|---|---|---|
| concentration | (Asp-Cys)₂ | (Cys-Asp)₂ | (Lys-Cys)₂ | (Cys-Lys)₂ | (Ala-Cys)₂ | (Pro-Cys)₂ |
| 50 mM | 0.042 | 0.039 | 0.047 | 0.077 | 0.039 | 0.118 |
| 25 mM | 0.039 | 0.279 | 0.045 | 0.067 | 0.037 | 0.762 |
| 10 mM | 1.301 | 1.523 | 0.556 | 0.092 | 1.129 | 1.379 |
| 5 mM | 1.453 | 1.702 | 0.946 | 0.390 | 1.052 | 1.539 |
| 0 mM | 1.279 | 1.279 | 1.279 | 1.279 | 1.279 | 1.279 |

With decreasing concentrations of cysteine, the molar ratio of dipeptide to cysteine required for full stabilization decreased down to the lowest measured ratio of 1:10. This is depicted in Table 3, for the results of turbidity measurements with the 25 mM cysteine solution.

TABLE 3
Turbidity of a 25 mM cysteine solution mixed with various concentrations of Cys-
dipeptides. Turbidity was measured at 600 nm (AU) after 4.5 hours

| Cys-Peptide | | | | | | |
|---|---|---|---|---|---|---|
| concentration | (Asp-Cys)₂ | (Cys-Asp)₂ | (Lys-Cys)₂ | (Cys-Lys)₂ | (Ala-Cys)₂ | (Pro-Cys)₂ |
| 50 mM | 0.040 | 0.040 | 0.047 | 0.084 | 0.050 | 0.042 |
| 25 mM | 0.038 | 0.038 | 0.042 | 0.068 | 0.037 | 0.040 |
| 10 mM | 0.039 | 0.037 | 0.041 | 0.050 | 0.037 | 0.039 |
| 5 mM | 0.038 | 0.081 | 0.039 | 0.044 | 0.039 | 0.078 |
| 0 mM | 0.122 | 0.122 | 0.122 | 0.122 | 0.122 | 0.122 |

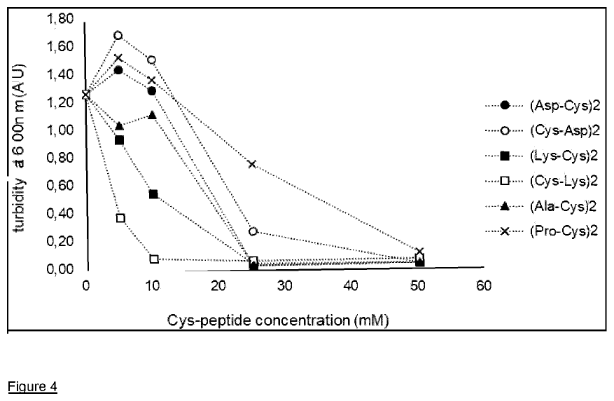

The results of Table 2 and 3 are visualized in FIG. 4 and Figure.

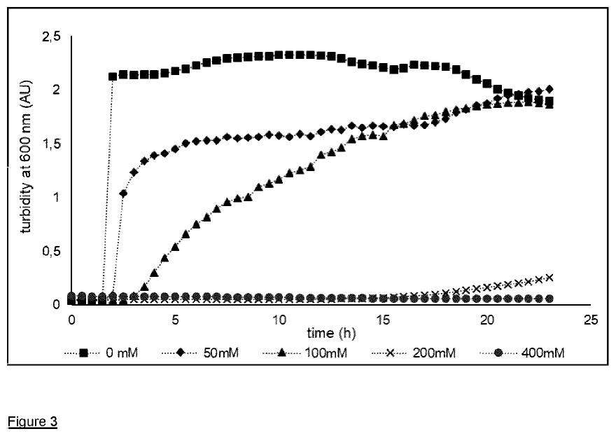

FIG. 3 shows stabilizing effect against oxidative precipitation provided by addition of various concentrations of Cys-peptides to a 50 mM cysteine solution.

FIG. 4 shows stabilizing effect against oxidative precipitation provided by addition of various concentrations of Cys-peptides to a 25 mM cysteine solution.

### Example 6: Stabilization of 50 mM Cysteine Solutions Against Precipitation by Addition of Reduced and Oxidized Cys-Dipeptides Under Oxidizing Conditions

To evaluate if the above described stabilization is also possible by addition of reduced Cys-dipeptides (=free thiol), the same experimental setup as described in example 4 was chosen. In this case, various concentrations of the reduced dipeptide Cys-Gly and of the oxidized dipeptide (Cys-Gly)₂ were added to a 50 mM cysteine solution. Both Cys-peptides were able to prevent precipitation from the cysteine solutions after addition of H₂O₂. The oxidized dipeptide (Cys-Gly)₂ was more efficient in preventing precipitation as precipitation could already be completely suppressed at a peptide concentration of 25 mM, whereas complete stabilization with the reduced dipeptide Cys-Gly was only obtained at 50 mM. The results are summarized in Table 4.

TABLE 4
Turbidity of a 50 mM cysteine solution mixed with various
concentrations of reduced Cys-Gly and oxidized (Cys-Gly)₂.
Turbidity was measured at 600 nm (AU) after 4.5 hours

| Cys-Peptide | | |
|---|---|---|
| concentration | Cys-Gly | (Cys-Gly)₂ |
| 50 mM | 0.04 | 0.04 |
| 25 mM | 0.26 | 0.04 |
| 10 mM | 0.37 | 0.07 |
| 5 mM | 0.58 | 1.07 |
| 0 mM | 1.32 | 1.32 |

1-15. (canceled)

16. A method of stabilizing a solution of cysteine against oxidative precipitation, the method comprising:
adding a dipeptide comprising solely alpha-peptide-bonds with one amino acid being cysteine (Cys) to the solution of cysteine, wherein the dipeptide is a Xxx-Cys dipeptide or a Cys-Xxx dipeptide, wherein Xxx is a natural amino acid.

17. The method of claim 1, wherein the natural amino acid Xxx is selected from the group consisting of glycine (Gly), alanine (Ala), serine (Ser), proline (Pro), aspartic acid (Asp), glutamic acid (Glu), lysine (Lys), and arginine (Arg).

18. The method of claim 17, wherein the natural amino acid Xxx is selected from the group consisting of alanine (Ala), proline (Pro), aspartic acid (Asp), and lysine (Lys).

19. The method of claim 16, wherein the solution of cysteine has a pH-value at 25° C. of at least 5.

20. The method of claim 16, wherein a molar ratio of the oligopeptide bound cysteine to the free cysteine is from 0.1 to 10.

21. The method of claim 16, wherein the dipeptide is in a reduced state with a free thiol.

22. The method of claim 16, wherein the dipeptide is in an oxidized state being disulfide bonded.

23. The method of claim 16, wherein the solution of cysteine is a culture medium for culturing cells.

24. The method of claim 23, wherein the cells are selected from the group consisting of CHO cells, COS cells, VERO cells, BHK cells, HEK cells, HELA cells, AE-1 cells, insect cells, fibroblast cells, muscle cells, nerve cells, stem cells, skin cells, endothelial cells, and immune cells.

25. The method of claim 23, wherein the immune cells are N K cells, T-cells, or hybridoma cells.
